(19) United States
(12) Patent Application Publication
(10) Pub. No.: US 2018/0177449 A1
(43) Pub. Date: Jun. 28, 2018

(54) FOOT MUSCLE BIOFEEDBACK UNIT

(71) Applicant: Penelope Jane Latey, Naremburn, New South Wales (AU)

(72) Inventor: Penelope Jane Latey, Naremburn, New South Wales (AU)

(21) Appl. No.: 15/577,154

(22) PCT Filed: Jun. 1, 2016
(86) PCT No.: PCT/AU2016/050437

§ 371 Date: Nov. 27, 2017

(30) Foreign Application Priority Data
Jun. 1, 2015 (AU) 2015902092

(51) Int. Cl. A61B 5/00; A61B 5/11; A61B 5/103

(52) U.S. Cl. CPC A61B 5/486; A61B 5/1107; A61B 5/1114; A61B 5/1036; A61B 2503/10; A61B 5/1038; A61B 5/74; A61B 2505/09; A61B 2562/0247; A61B 5/6807

(57) ABSTRACT

The present invention relates broadly to a device for evaluating the mobility of a foot arch of a person. The device includes a base formed from two separate base members. The first base member includes a first zone configured to receive a foot of the or user to be evaluated. Position sensors in the form of a plurality of contact pressure sensor are mounted within the first zone of the first base member for detecting whether the foot to be evaluated is in a desired position relative to the first base member. The first base member includes a biofeedback sensor engaged or contacted by at least a portion of the foot arch. The biofeedback sensor includes an inflatable bladder mounted to the first base member and configured so that changes to the configuration of the foot arch cause a corresponding change to the configuration of the bladder.

### FIELD OF THE INVENTION

The present invention relates broadly to foot arch mobility and the relationship between foot arch mobility and more specifically the intrinsic or plantar muscles of a human foot.

### BACKGROUND OF THE INVENTION

The following discussion of the prior art has been provided in order to place the invention in an appropriate technical context and allow the advantages of it to be more fully appreciated. However, any discussion of the prior art throughout the specification should not be considered as an express or implied admission that such prior art is widely known or forms part of common general knowledge in the field.

Strong healthy mobile feet are important for maintaining a healthy and active lifestyle. However, many people suffer from problems related to their feet which significantly affect their everyday activities such as walking and climbing stairs. For some, these feet related problems provide great discomfort and pain. These people necessarily compensate for this discomfort by changing their gait and/or vertical posture, with a flow-on effect leading to other more serious posture related problems such as lower back pain.

There are many causes of compromised foot function including foot specific pathologies, systemic diseases and obesity, many of which are exacerbated with ageing.

Systemic diseases such as diabetes, peripheral arterial disease, gout, psoriasis, collagen disorders and rheumatoid arthritis can all lead to various foot-related problems. Various genetic disorders such as multiple sclerosis, muscular dystrophy, neural tube deficits and Charcot Marie Tooth syndrome can also lead to foot problems. Body weight has also been found to be a cause for many foot problems. For those who are obese, upright movement can be severely compromised or impossible. In severe cases, obesity can lead to a person being unable to physically touch or see their own feet.

A regular exercise regimen is not easy to undertake by those suffering from systemic diseases such as diabetes or by those who are obese. This failure to exercise their leg and foot muscles further compounds their postural and other problems. Moreover, this can be something of a vicious cycle, whereby diminishing mobility and exercise lead to loss of nerve sensation, muscle atrophication and diminished circulation in the feet, which compound to further diminish mobility, sensation and the ability to undertake corrective exercise. In extreme cases, this can lead on to the development of chronic sores and can even require amputation of toes or feet.

Even active healthy people are not, for the most part, consciously aware of the operation of their intrinsic foot muscles, how to exercise them effectively, or the impact that these muscles have on the foot arch and upright posture. Indeed, even specialist medical practitioners, podiatrists and physiotherapists are only beginning to understand the complex biomechanical interrelationships between foot arch mobility and the intrinsic muscles of the foot, and the wider significance of them.

Devices have hitherto been developed to measure foot arch shape and configuration, in an attempt to explore these relationships. However, known devices of this type have suffered from a number of inherent disadvantages or limitations, including in connection with the inability to adapt readily to feet of different size and shape, the ability to allow easy calibration, the ability to provide consistent and accurate readings, and the ability to provide real-time feedback to the patient or user. Furthermore, these earlier devices have primarily been configured to assess foot arch drop in terms of changes in height, as opposed to foot arch spring or lift in relation to the foot arch configuration as a whole.

### SUMMARY OF THE INVENTION

According to a first aspect of the invention there is provided a device for evaluating the mobility of a foot arch of a person, the device including:

a base for supporting a foot;

sensing means associated with the base and arranged to detect a change in configuration of the foot arch predominantly occurring in response to intrinsic foot muscle activity within the supported foot, the sensing means being in contact with the foot arch;

position sensing means associated with the base and independent of the sensing means, said position sensing means configured to detect whether the supported foot is in a desired functionally effective position relative to the base;

a feedback module operatively associated with the sensing means for providing feedback to the person indicative of the change in the configuration of the foot arch predominantly occurring in response to the intrinsic foot muscle activity within the foot.

It will be appreciated that the sensing means can also be used to detect a lack of change in foot arch configuration or a lack of intrinsic foot muscle activity. Therefore, references throughout the specification to sensing or detecting should be understood to encompass the sensing or detection of an absence of such change or muscle activity. It should also be appreciated that the sensing means may be arranged to detect changes in foot arch configuration either directly, or indirectly on the basis of related foot configuration parameters, such as the positional relationship between the heel and toes.

It is to be understood that the change in configuration of the foot arch occurs predominantly in response to intrinsic foot muscle activity although extrinsic foot muscles may also contribute to this change in the foot arch configuration

In some preferred forms, the feedback module is configured to provide short-term or substantially real-time feedback to the person. In other forms, the feedback module is configured to enable feedback data to be collected and stored such that improvements or other changes to foot arch mobility can be evaluated over time. That is, the feedback module can be configured to provide long term feedback to the person. In certain embodiments, the feedback module is configured to provide both substantially real-time feedback and long term feedback.

Those skilled in the art will appreciate that the phrase “ foot muscle activity” refers to both relaxing and contracting of one or more muscles of or associated with the foot, and any associated arch drop or arch lift. It will also be appreciated that the intrinsic foot muscles do not work in isolation, but rather form part of the complex anatomy of the human foot. In particular, the intrinsic foot muscles work in combination with ligaments, tendons, bones and extrinsic foot muscles associated with the lower leg and ankle. Advantageously, however, the present invention, at least in preferred embodiments, enables the effect of predominantly the intrinsic foot muscle activity on foot arch mobility, particularly arch lift and arch drop, to be evaluated and assessed.

In certain preferred embodiments, the invention is formed as a stand-alone device suitable for stationary use in a domestic or clinical environment, for assessment, training and rehabilitation of the foot muscles. In other preferred forms, the device can be configured to be worn by a user while walking and may, for example, be incorporated into footwear such as a shoe.

Preferably, the sensing means is mounted on the base such that the sensing means is engagable or contacted by at least a portion of the foot arch. The sensing means is preferably a biofeedback sensor. Preferably, the biofeedback sensor includes a flexibly and/or elastically deformable body disposed such that changes to the configuration of the foot arch cause a corresponding change to the configuration of the elastically deformable body. In one preferred form, the elastically deformable body is an inflatable bladder. The bladder is preferably arranged on the base such that at least a portion of the sole of the supported foot is in direct face-to-face abutment with an operatively upper surface of the bladder.

In some preferred forms, the biofeedback sensor can be configured to detect a change in the overall configuration of the foot arch. In other forms, the biofeedback sensor can be configured to detect changes in one of more of the longitudinal arches (including the medial and lateral arches) and/or the transverse arches of the foot.

In some embodiments, the biofeedback sensor includes a pressure sensor for detecting changes in pressure within the bladder corresponding to changes in the configuration of the foot arch. In other embodiments, the biofeedback sensor may additionally or alternatively include other forms of sensor, such as position sensors, displacement sensors, proximity sensors, accelerometers or the like.

The biofeedback sensor preferably includes a pump for inflating the bladder to a predetermined pressure. Preferably, the pump is a selectively operable hand pump, the pump being fluidly connectable to the bladder by a supply line. In one preferred form, the hand pump is an elastically squeezable bulb. In other embodiments, the bladder may be self-inflating, for example by means of an electric pump, an internal or remote source of pressurised gas, or mechanical bladder expansion elements. The bladder is preferably inflated by a gas such as air, but could alternatively be inflated or pressurised by a liquid or gel.

The pressure sensor preferably includes a calibration mechanism to account for various shapes and sizes of feet. Preferably, the calibration mechanism includes a release valve adapted to calibrate the pressure in the bladder to a control pressure after inflation to the predetermined pressure. The control pressure preferably enables the bladder and the associated sensors to detect both arch lift and arch drop.

Preferably, the feedback module includes an output device for providing the feedback to the user. The output device preferably provides the feedback as one or more output signals. Preferably, the output signals include one or more of audio, visual and/or tactile signals. The feedback module is preferably in communication with the pressure sensor whereby the pressure detected by the pressure sensor is converted to the output signal.

In certain preferred embodiments, the output device has a display for displaying the feedback to the user as a visual signal. In some embodiments, the visual signal is a variable signal which changes in shape and/or size in response to changes in the configuration of the foot arch. Preferably, the change to the variable signal correlates approximately in both direction and magnitude to changes in the foot arch. For example, the variable signal may be a variable graphic which increases in size as the foot muscles cause the foot arch to lift and decreases in size as the foot arch drops. In one preferred form, the variable signal includes an array of light-emitting diodes (LEDs), each LED being configured to be in one of an “on” state or an “off” state, such that changes to the configuration of the foot arch can be indicated by a change in the number of indicia in the on state. For example, in one embodiment, as the intrinsic foot muscles cause the foot arch to lift, the number of indicia in the on state increases, or vice versa. Similarly, as the intrinsic foot muscles cause the foot arch to drop, the number of indicia in the on state decreases, or vice versa.

It will be appreciated that the feedback is not limited to those output signals listed above, but may be any suitable signal for providing feedback to the user. For example, in one embodiment, an audio signal progressively increases in amplitude and/or frequency as the foot arch lifts in response to foot muscle activation, and progressively decreases in amplitude and/or frequency as the foot arch drops. These embodiments are advantageous for users who are visually impaired. In other embodiments, a tactile feedback mechanism may be arranged to provide, for example, feedback indicative of foot arch changes in the form of pressure applied to the skin of the user.

In some embodiments, the feedback may be progressive or analogue in nature, while in other embodiments it may be step-wise. In yet other embodiments, the feedback mechanism may simply provide a threshold “on” or “off” signal to indicate that a predetermined degree of change in foot arch lift or configuration has occurred.

The position sensing means preferably includes one or more sensors, more preferably, pressure or contact sensors. In certain preferred embodiments, the one or more contact sensors include a heel sensor and at least one toe sensor. The position sensing means preferably includes two toe sensors. Preferably, the two toe sensors include a great toe sensor and a fifth toe sensor. In certain embodiments, the position sensing means may include a sensor for additional toes, optionally for each toe, or for other parts of the foot. It will of course be appreciated by those skilled in the art the position sensing means could be configured to detect other portions of the foot, rather than the heel and toes. For example, the position sensing means may be configured to detect the position of the ball of the foot relative to the base.

In some embodiments, the position sensors operate by sensing pressure applied by various parts of the foot. It will be appreciated, however, that other forms of position sensing may be used. For example, sensors based on electrical conductivity, electro• mechanical contact switches or even optical sensors could additionally or alternatively be used for this purpose. In some embodiments, pressure-based sensors may also be used to provide a quantitative indication of the pressure applied by the foot or toes at selected contact points, so as to provide additional information about weight distribution and foot muscle activity. In some embodiments, the same sensors are used to provide information about both foot position and weight or pressure distribution.

In certain embodiments, the heel sensor and/or the at least one toe sensor are selectively moveable relative to one another to accommodate various sizes of feet, as well as both left and right feet. The sensors are preferably movable to accommodate different feet lengths and widths. In one embodiment, the heel sensor is fixed in position relative to the base and the at least one toe sensor is selectively movable relative to the heel sensor. It will be appreciated that, in other preferred embodiments, the at least one toe sensor could be configured to account for various sizes of feet whilst being mounted in fixed relation relative to the base.

Each position sensor preferably has an associated position indicator for indicating whether or not the associated portion of the foot is in the desired position. In some embodiments, the position indicator is mounted on the base. In other embodiments, the position indicator is in communication with the feedback module, such that the feedback module indicates in use whether or not one or more of the heel, the great toe and the fifth toe of the supported foot are in their respectively desired positions relative to the base.

It will be appreciated that when the foot is initially in position and subsequently maintained in the desired position relative to the base, the user is more readily able to isolate the influence of the extrinsic foot muscles, and focus control on the intrinsic foot muscles, in order to change the configuration of the foot arch in a way that can be consistently and reliably monitored by the sensor mechanism. Accordingly, if the user becomes aware that their foot has moved from the desired position, he or she can readily return the foot to the desired position and recommence their intrinsic foot muscle exercises, having received positive feedback to confirm that the foot is back in the desired position. By knowing the position of the foot relative to the base, the person is therefore able to monitor and ‘feel’ the effect of their intrinsic foot muscle activity on the configuration of their foot arch.

Furthermore, it should also be understood that the position sensing means and the sensing means (e.g. the biofeedback sensor) can be used in combination to detect the relationship between foot arch configuration (and changes thereto) and the relative position of the heel and toes.

The device preferably further includes an input module for allowing control of the device and selection of options by the user. Preferably, the input module includes at least one selector switch for selecting between a right foot mode and a left foot mode. The input module preferably includes a calibration switch in communication with the release valve, the calibration switch being configured to allow the user to calibrate the pressure within the bladder in accordance with a predetermined calibration protocol. It will be appreciated that the input module can include various other suitable input mechanisms for allowing user data to be entered and/or to customise the device to the attributes of the user.

The device preferably includes a user interface comprising the feedback module and the input module. The user interface preferably includes a housing for housing both the input module and the feedback module. In certain embodiments, the user interface is connected to the sensing means via a control line for allowing communication between the sensing means, the feedback module and the input module. The control line is preferably flexible such that the user interface is movable relative to the base. The control line may be adapted to carry electronic, optical, pneumatic or other control signals from the sensing means. In other embodiments, the user interface is in wireless communication with the sensing means.

It will be appreciated that in some preferred embodiments, the feedback module and the input module are not housed in the one unit. For example, the display of the feedback module could be in the form of an electronic screen mountable on a wall or desk for easy viewing by the user and/or a clinician. Similarly, the input module could be in the form of a remote control-type device.

In certain embodiments, the device includes a processor having a memory for storing the information obtained from the sensing means and the position sensing means. Preferably, the device has a communication interface for interfacing the processor with an analysis tool such as a personal computer, the interface allowing information to be exchanged between the processor and the analysis tool. Preferably, the interface allows the information obtained from the sensing means and the position sensing means to be downloaded from the processor to the analysis tool. In certain embodiments, the communication interface is configured to allow wireless communication between the processor and the analysis tool.

Preferably, the base is configured to support both feet of a user in spaced apart side-by-side relation. The base preferably has a first zone for receiving the foot to be evaluated and a second zone for supporting the other foot of the person. In some embodiments, the base is a one-piece unit sized to receive both feet of the user. In other preferred embodiments, the base is formed from two or more separate base members wherein one base member defines the first zone and another base member defines the second zone. In some embodiments, two separate base members are connected to each other by a connecting means. The connecting means is preferably a hinge-type member which enables the two base members to be selectively moved between an operative or open configuration for supporting the feet of the user and a closed or collapsed position for transporting and storing the device. Preferably, the connecting means is configured to inhibit the relative movement of the two base members beyond the intended operational range.

It will be appreciated that in certain other embodiments, the base is sized and configured to support only the foot which is to be evaluated. For example, the base could be configured as an insole for a shoe. In such embodiments, the user interface could be configured to be in the form of, for example, a mobile or cell phone, a tablet, or a wrist watch-type unit in wireless communication with the sensing means.

The sensing means and position sensing means are both preferably associated with the first zone of the base. Preferably, the first and second zones are configured to support the left or right foot of a user.

It should also be appreciated that in some embodiments, a pair of the devices may be incorporated respectively into first and second zones, both zones being operational, so as to permit independent evaluation of the mobility of the arches or both feet of the user simultaneously.

According to a second aspect, the invention provides a method of evaluating the mobility of a foot arch of a person, said method including the steps of:

supporting a foot of the person on a base;

providing sensing means associated with the base and arranged to detect a change in configuration of the foot arch predominantly occurring in response to intrinsic foot muscle activity within the supported foot, the sensing means being in contact with the foot arch;

providing position sensing means associated with the base and independent of the sensing means, said position sensing means configured to detect whether the supported foot is in a desired functionally effective position relative to the base; and

using a feedback module operatively associated with the sensing means to provide feedback to the person indicative of the change in the configuration of the foot arch predominantly occurring in response to the intrinsic foot muscle activity within the foot.

### BRIEF DESCRIPTION OF THE DRAWINGS

A preferred embodiment of the invention will now be described, by way of example only, with reference to the accompanying drawings in which:

FIG. 1 is schematic view of a device for evaluating the mobility of a foot arch of a person according to an embodiment of the invention;

FIG. 2 is a schematic view of the device of FIG. 1 showing a person using the device to evaluate the foot muscles in their right foot;

FIG. 3 is a perspective view of a first base member of the device;

FIG. 4 is a perspective view of a second base member of the device;

FIG. 5 is an enlarged view of a biofeedback sensor of the device;

FIG. 6 is an enlarged perspective view of a user interface and hand pump of the device;

FIG. 7 is a perspective view of the user interface in which all outputs are off;

FIG. 8 is a perspective view of the user interface in which the position indicator for the heel sensor is on;

FIG. 9 is a perspective view of the user interface in which the position indicators for the heel and the great toe sensors are on;

FIG. 10 is a perspective view of the user interface in which the position indicators for the heel, great toe and fifth toe sensors are on, and the output LEDs are showing that the foot arch is in the neutral configuration;

FIG. 11 is a perspective view of the user interface in which the output LEDs are showing that the foot arch has lifted from the neutral configuration;

FIG. 12 is a perspective view of the user interface in which the output LEDs are showing that the foot arch has dropped from the neutral configuration;

FIG. 13 is a perspective view of the user interface with its cover removed to show the internal components; and

FIG. 14 graphically illustrates comparative screen displays for different users of the device in a preferred embodiment.

### PREFERRED EMBODIMENT OF THE INVENTION

Referring to the drawings, the invention provides a device 1 for evaluating the mobility of a foot arch 2 of a person. In a preferred embodiment the device 1 includes a base 3 formed from two separate base members 4 and 5. The first base member 4 is in the form of a substantially planar member having an upper surface which defines a first zone 6. The first zone 6 is configured to receive a foot 7 of the person or user to be evaluated. The first zone 6 is configured to receive either the right or left foot of the user. That is, the user can evaluate one foot and then swap feet to evaluate the other foot.

The second base member 5 is also in the form of a substantially planar member having an upper surface which defines a second zone 8 for supporting the other foot of the person.

The first and second base members (4, 5) are constructed to be of substantially the same thickness ‘T’. It will be appreciated that the use of paired base members having substantially the same thickness advantageously provides a neutral balanced foot support platform for the person, in use. That is, the paired base members enable the weight of the person to be distributed substantially evenly over both feet.

Position sensors in the form of a plurality of contact pressure sensors are mounted within the first zone 6 of the first base member 4 for detecting whether or not the foot 7 to be evaluated is in a desired position relative to the first base member 4. The contact sensors of this embodiment include a heel sensor 9 and four toe sensors. The toe sensors include two great toe sensors (10a, 10b) and two fifth toe sensors (11a, 11b). When the right foot of the user is being evaluated, the left great toe sensor 10a and the right fifth toe sensor 11b will be activated, and the right great toe sensor 10b and left fifth toe sensor 11a deactivated. In contrast, when the left foot of the user is being evaluated, the right great toe sensor 10b and the left fifth toe sensor 11a will be activated, and the left great toe sensor 10a and right fifth toe sensor 11b deactivated.

In another embodiment, there are only two toe sensors, wherein one acts as the great toe sensor and the other as the fifth toe sensor, depending on whether the right or left foot is being evaluated. That is, when the right foot of the person is being evaluated one of the two toe sensors will act as the great toe sensor 10 and the other toe sensor will act as the fifth toe sensor 11. In such embodiments, when the left foot of the person is being evaluated, the roles of the two toe sensors will swap.

A heel support 12 is mounted at one end of the first base member 4. The heel sensor 9 is fixedly mounted to the first base member 4 adjacent the heel support 12.

The first base member 4 has two mounting formations in the form of slots 13 in which the great toe sensor 10 and fifth toe sensor 11 are mounted, respectively. The toe sensors (10, 11) are independently slidably movable along the respective slots 13 such that the toe sensors can be moved relative to the heel sensor 9 to suit different foot 7 lengths and widths.

A cover (not shown) is preferably fitted over the first base member to cover the heel and toe sensors. The cover has a cut-out portion through which sensing means can pass for exposure and contact with a foot arch. The cover may be removable to provide access to the toe sensors whereby the user can slide the sensors along the slots 13 to suit the size and shape of the foot which is being evaluated. In other embodiments, an external slider (not shown) is connected to each toe sensor for sliding the sensors along the slots such that the cover need not be removed.

As will be described in greater detail below, the heel sensor 9, great toe sensor 10, and fifth toe sensor 11 each have an associated position indicator in the form of LEDs 14A, 14B, and 14C, respectively, for indicating whether or not the associated portion of the foot 7 is in the desired position. It will be appreciated that when the heel, the great toe and little toe are in the desired position, it follows that the foot 7 is in the desired position relative to the base.

Accordingly, if the LEDs 14 indicate that the foot 7 has moved from the desired position, the user can correct the position of the foot 7 and recommence foot muscle exercises once they have received positive confirmation from the LED indicators that the foot 7 is back in the desired position. By knowing the position of the foot 7 relative to the first base member 4, the person is able to monitor and ‘feel’ the effect of their foot muscle activity on the configuration of their foot arch 2.

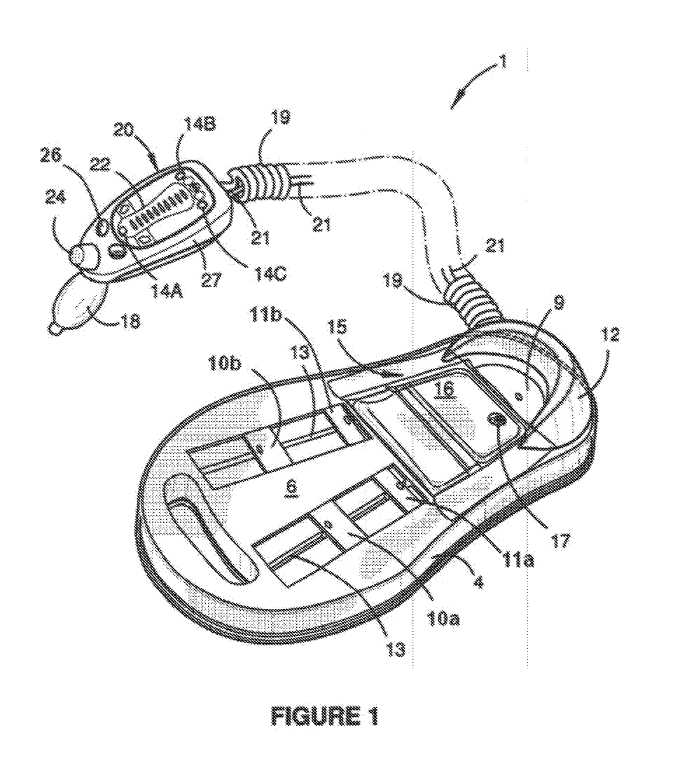

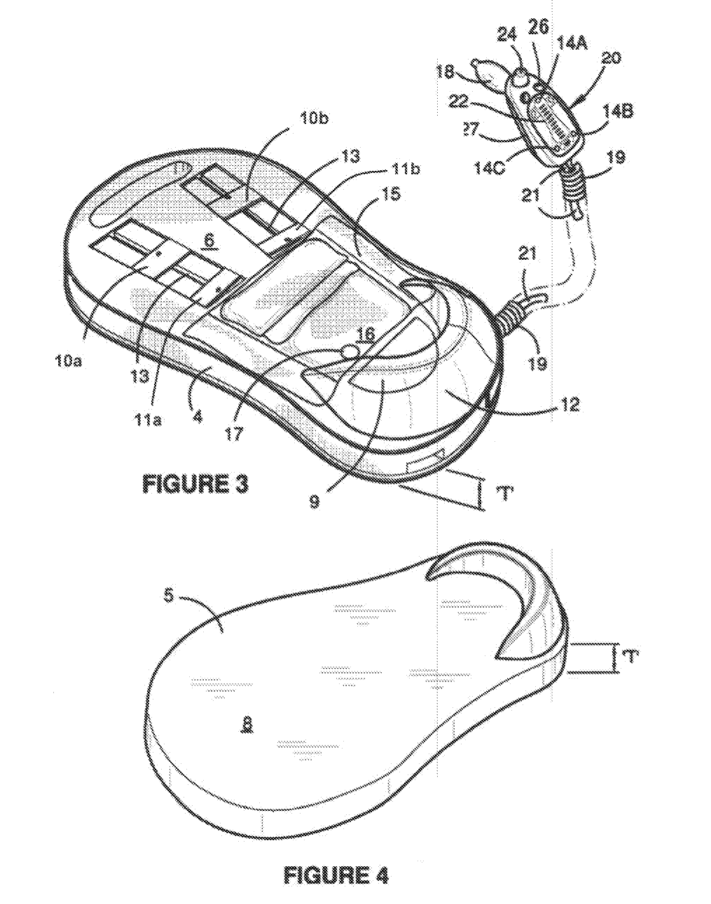

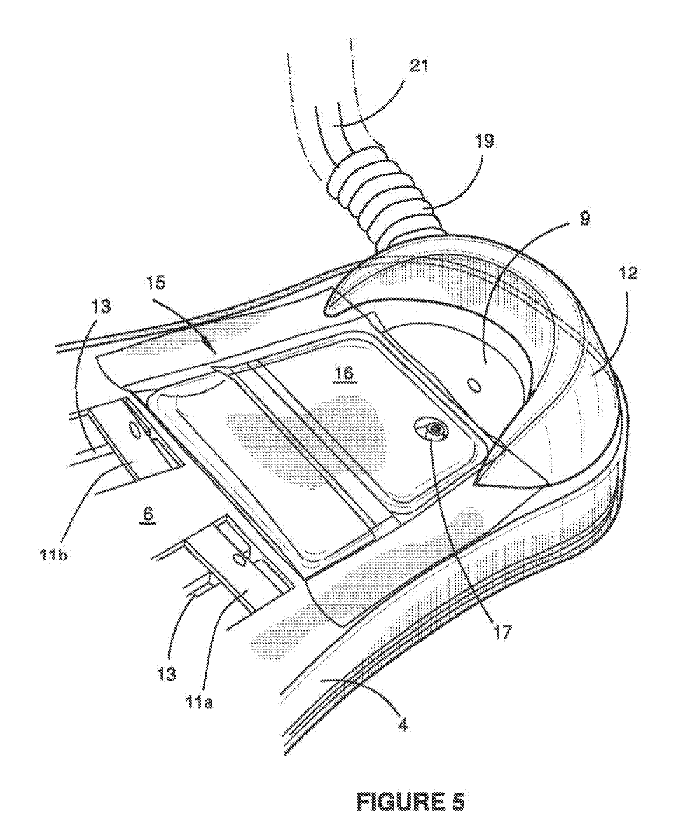

As most clearly shown in FIGS. 1, 3 and 5, the sensing means of this example is in the form of a biofeedback sensor 15 mounted on the first base member 4. When the foot 7 is supported on the first base member 4, the sensor 15 is engaged or contacted by at least a portion of the foot arch 2. The biofeedback sensor 15 in this embodiment includes an inflatable bladder 16 which is elastically deformable in use. The bladder 16 is mounted on the first base member 4 such that it is disposed generally intermediate the heel sensor 9 and the two toe sensors (10, 11).

The bladder 16 is configured such that changes to the configuration of the foot arch 2 cause a corresponding change to the configuration of the bladder 16. Provided the foot 7 is maintained in the correct position and in contact with the base (confirmed by the indicator LEDs as previously described), the changes to the configuration of the foot arch 2 which are detected are those predominantly occurring in response to intrinsic foot muscle activity within the supported foot. It will be appreciated that such changes to the configuration of the bladder 16 cause a corresponding change in pressure within the bladder 16. The biofeedback sensor 15 includes a pressure sensor 17 for detecting these changes in pressure within the bladder 16.

To inflate the bladder 16, the biofeedback sensor 15 includes a selectively operable hand pump in the form of an elastically squeezable bulb pump 18. The pump 18 is connected to the bladder 16 by a supply line 19. As will be described in greater detail below, the pump 18 is used to initially inflate the bladder 16 to a predetermined pressure. In an alternative embodiment the pump may be mounted within the first base member 12 eliminating the need for the supply line 19. The pump may be manually activated for inflation of the bladder 16 or may automatically activate relying on an electric motor (not shown) onboard the base member 12 and designed to inflate the bladder to a pressure preset by the user.

A multi-position pressure range selector switch, preferably a four-position switch (not shown) is located at the rear of the first base member 4. The four-position switch is operatively associated with the bladder 16 and the pump 18 of the biofeedback sensor 15 and is configured to allow selection of one of four different pressure ranges. For example, the pressure ranges can be 0-40 cmH₂0, 0-20 cmH₂0 and 0-5 cmH₂0. It will be appreciated that the selection of various pressure ranges provides for various sensitivities such that the range can be selected to suit the change in foot arch 2 configuration of a particular person. In addition, the multi-position pressure range selector switch can be used to identify slow leaks in the bladder 16 by selecting the 0-5 cmH₂0 pressure range. The multi-position pressure range selector switch can also be used to “zero” the device when the bladder 16 is inflated with no foot present.

Calibrating means in the form of a pressure release valve (not shown) is operatively associated with the pressure sensor 17. The release valve is adapted to calibrate the pressure in the bladder 16 to a control pressure after initial inflation to the predetermined pressure. The control pressure advantageously enables the bladder 16 to detect both arch lift and arch drop.

A user interface 20 is in operative communication with the biodfeedback sensor 15 and the position sensing means. The user interface 20 includes an input module and a feedback module.

The user interface 20 is in this embodiment connected to the first base member via a control line 21 to enable communication between the position sensing means, the biofeedback sensor 15, the input module and the feedback module. The control line 21 is flexible such that the user interface 20 is movable relative to the first base member 4. In an alternative embodiment the communication between the user interface 20 and the biofeedback sensor 15 (and position sensing means) is provided wirelessly. For example, the interface 20 may communicate with the sensor 15 (and position sensing means) across a Bluetooth link with the devices appropriately paired.

In the illustrated embodiment, the feedback module is operatively associated with the biofeedback sensor 15 to provide substantially real-time feedback to the user. The feedback provided by the feedback module is indicative of the changes in pressure within the bladder 16 of the sensor mechanism, which in turn are indicative of changes in the configuration of the foot arch 2 predominantly occurring in response to foot muscle activity associated with the foot 7.

The feedback module includes an output device in the form of a display 22 to provide a visual signal which correlates to the changes to the foot arch 2 configuration.

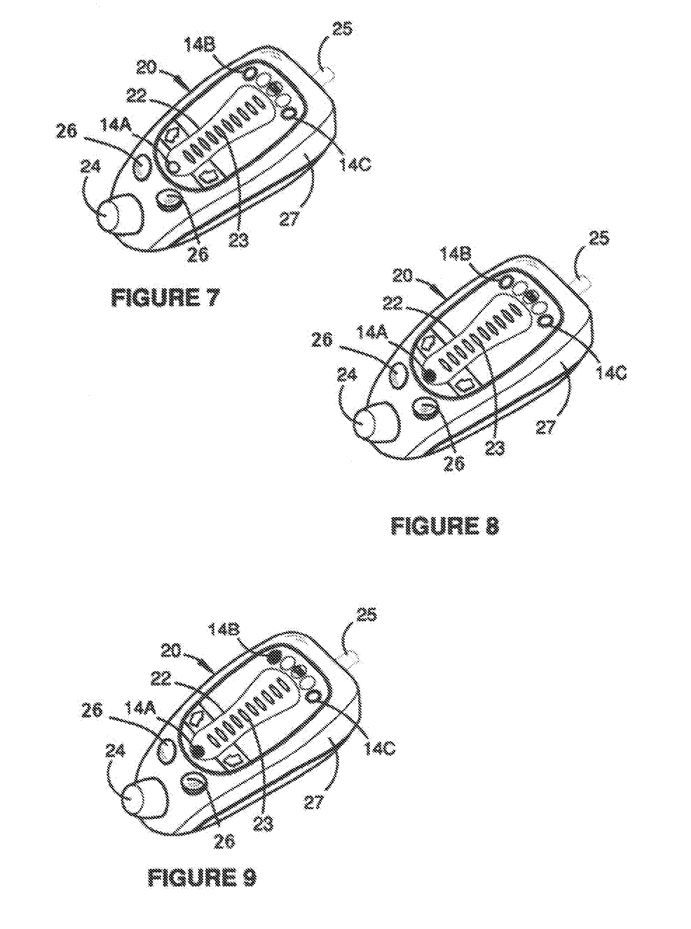

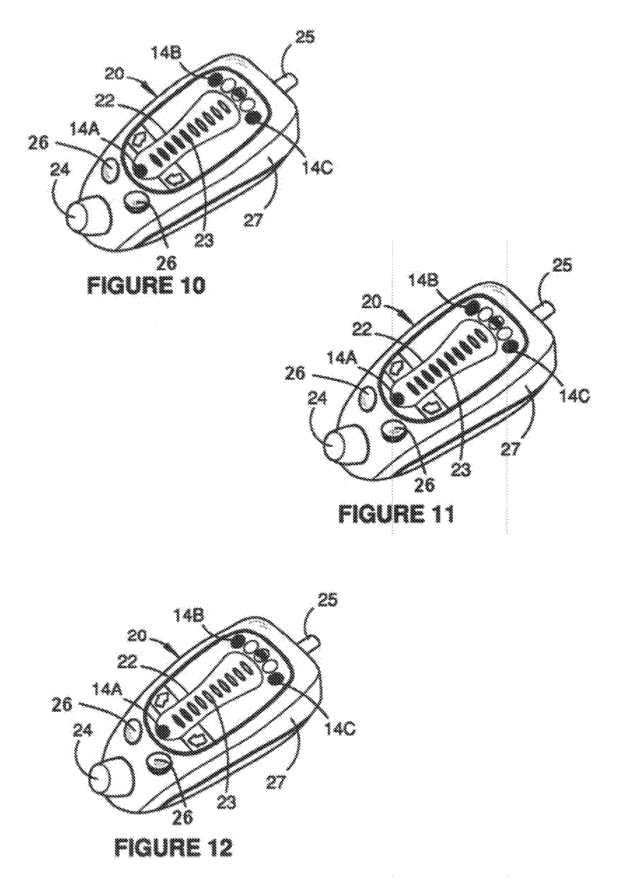

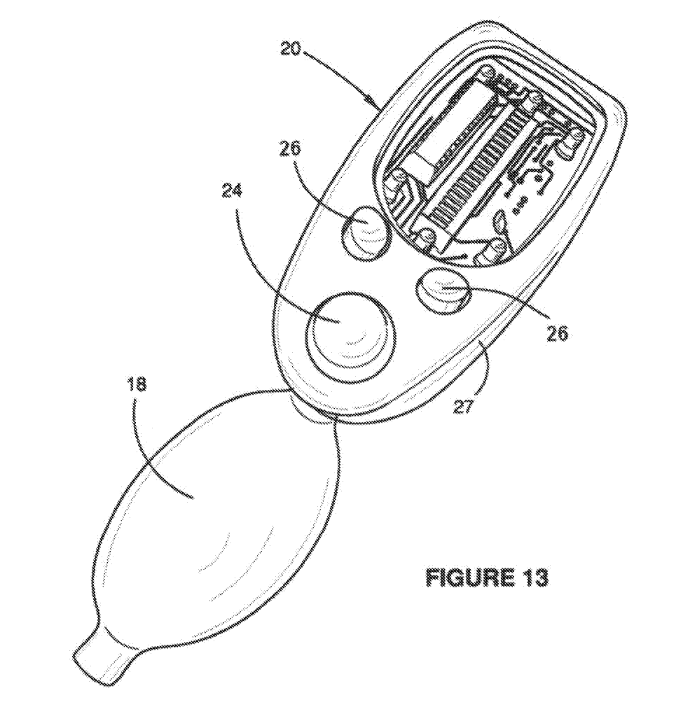

As most clearly shown in FIG. 7, the visual signal is preferably formed from a linear array of light-emitting diodes (LEDs) 23. Each LED 23 is configured to be in either an “on” state or an “off” state. The changes to the configuration of the foot arch 2 are thus indicated by a change in the number of LEDs 23 which are in the “on” state. For example, as shown in FIG. 11, as the foot muscles cause the foot arch 2 to lift, the number of LEDs in the “on” state progressively increases. That is, as the height of the foot arch 2 increases, the number of LEDs 23 in the on state also increases. Similarly, as shown in FIG. 12, the number of LEDs 23 in the on state decreases as the foot muscles cause the foot arch 2 to drop. By receiving such real-time feedback, the user gradually gains a ‘feel’ for in particular their intrinsic foot muscle activity and the direct effect that this muscle activity has on the configuration of their foot arch 2.

It will be appreciated that it is advantageous for the user interface to be movable relative to the base, as it enables less mobile people or those physically unable to see their own feet to position the user interface 20 such that they can receive substantially real-time feedback indicative of their foot muscle activity.

The input module includes an input device for allowing user selections by the person. The input device includes a selector switch 24 for selecting between a right foot mode and a left foot mode. The input device also includes a calibration switch or button 25 which is operatively connected to the pressure relief valve to allow the user to calibrate the pressure in the bladder 16 to the control pressure, as part of a predetermined calibration protocol. The input device also includes input buttons 26 to allow the user to operate the device, enter user specific information and/or reset the device.

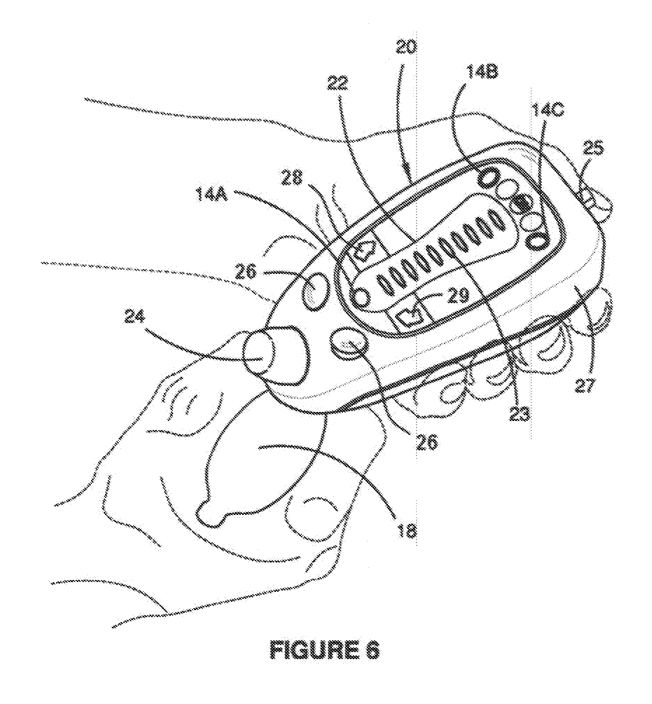

As most clearly shown in FIG. 6, the user interface 20 includes a housing 27 for housing both the input module and the feedback module. The housing 27 also includes the LEDs 14 associated with the heel sensor 9, great toe sensor 10 and fifth toe sensor 11. Again, this enables real-time feedback to the person of whether or not their foot 7 is in the desired position whilst conducting their foot muscle exercises.

In an alternative embodiment the user interface is in the form of a mobile device such as a mobile or cell phone, or tablet. The input module in this embodiment is in the form of a touchscreen associated with the mobile device. The feedback module is in the form of a screen display associated with the mobile device. The mobile device may include a memory storing software for implementation of the device 1 via a processor associated with the mobile device. The software may take the form of an application or App which is downloaded to the mobile device. The mobile device may also be configured to save and/or send data associated with the sensing means for review by a practitioner, clinician or other specialist.

In use, the first base member 4 and second base member 5 are arranged next to each other to support both feet of a user in spaced side-by-side relation. The device 1 can be used with a person in seated, standing or standing leg bend positions. In this regard, it is recommended that the person initially commence in the seated position, before progressing to the more advanced standing positions. It will be appreciated that these various positions provide non-weight bearing, weight bearing and stressed positions, respectively. In each of these positions, the device can provide feedback on the ability of the user to engage their foot muscles to control arch deformation as a ratio of flexibility (arch drop), range of motion (arch lift) and endurance (ability to repeat arch lift). Furthermore, the use of various weight-bearing and non-weight-bearing positions in combination with pressure sensors for detecting the pressure exerted by various portions of the foot 7 on the base enables the effect of load or gravity on a person's ability to activate their foot muscles and their associated foot arch 2 configuration to be evaluated.

Before the user places their foot 7 on the first base member 4, the device is turned on and the user selects either the right foot mode or the left foot mode via the switch 24. With no foot present, the device calibrates itself as indicated by a continuous tone for approximately two seconds.

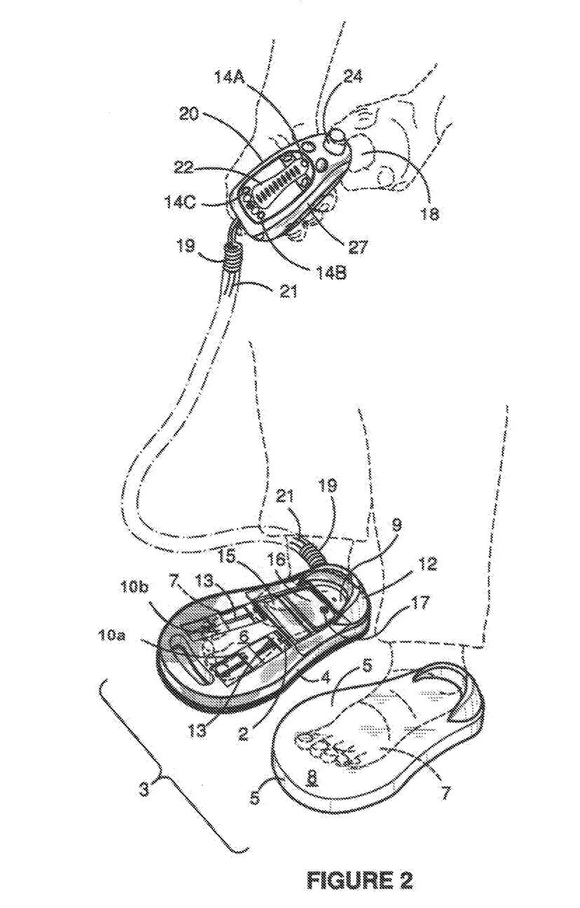

Once the person has placed their feet on the base members, the person takes the user interface in one hand and the hand pump 18 in the other hand, as shown in FIGS. 2 and 6. The left button 26 is then pressed to enable the pressure sensors. This initiates a calibrating routine in which the foot sensors are set for sensing the great toe and fifth toe, as required, over a period of approximately two seconds. This calibrating routine may by indicated by an audible signal such as a chirping tone.

The user then observes the LEDs 14 on the user interface 20 to verify that their heel, great toe and fifth toe are in the desired position.

As shown in FIG. 7, each of the position indicating LEDs 14 are in the “off” state before the foot 7 is placed in the desired position. The user will typically move their foot 7 in order to first place their heel in the desired position. Once the heel has been placed in the desired position, the heel sensor position indicator 14A will light up as shown in FIG. 8. Similarly, as shown in FIG. 9, the great toe sensor position indicator 14B will light up once the great toe is moved to the desired position and finally, the fifth toe position indicator 14C will illuminate to confirm when the fifth toe is in position (see FIG. 10). The LEDs 14 may also change colour in response to pressure applied by the heel and toes so that over or under inflation of the bladder 16 can be controlled.

Referring now to FIG. 10, once the three LEDs 14 indicate that the foot 7 is in the desired position in firm contact with the base, the user relaxes the foot 7 and then repeatedly squeezes the hand pump 18 in order to inflate the bladder 16 to the predetermined pressure. In the alternative embodiment where the pump is onboard the first base member 4, the bladder 16 is automatically inflated to the required pressure set by the user. At this stage, the person again observes the LEDs 14 to confirm that their foot 7 is still in the desired position.

For the manually inflated bladder 16, the calibration button 25 is now pressed to calibrate the pressure within the bladder to the control pressure. The user interface includes LEDs in the form of arrows (28, 29) for indicating under- or over-inflation of the bladder 16. Once the pressure in the bladder 16 has been calibrated, a predetermined number of the output display LEDs 23 of the feedback module are turned “on” as shown in FIG. 10. This predetermined number of LEDs represents that person's neutral foot arch 2 configuration. A further and final check of the foot position LEDs 14 can now be made to confirm that the foot 7 is in the desired position, prior to commencing to exercise their foot muscles.

With their foot 7 in the desired position, the user is able to activate their foot muscles with a view to changing the configuration of their foot arch 2. As the user begins alternately to contract and relax their foot muscles, their foot arch 2 will lift or drop accordingly. As shown in FIGS. 11 and 12, this lift or drop will be shown as increases and decreases in the number of LEDs 23 which are “on”, relative to the neutral foot arch 2 configuration. That is, in FIG. 11 an additional three LEDs have turned on, indicating that the foot arch 2 has lifted by a corresponding extent from the neutral foot arch 2 configuration. In contrast, in FIG. 12 only two LEDs are on, indicating that the foot arch 2 has dropped from the neutral foot arch 2 configuration. Alternatively the visual feedback may be replaced or complemented with audio feedback indicative of the user's foot arch configuration in response to foot muscle activity. The audio feedback is particularly helpful for the sight impaired.

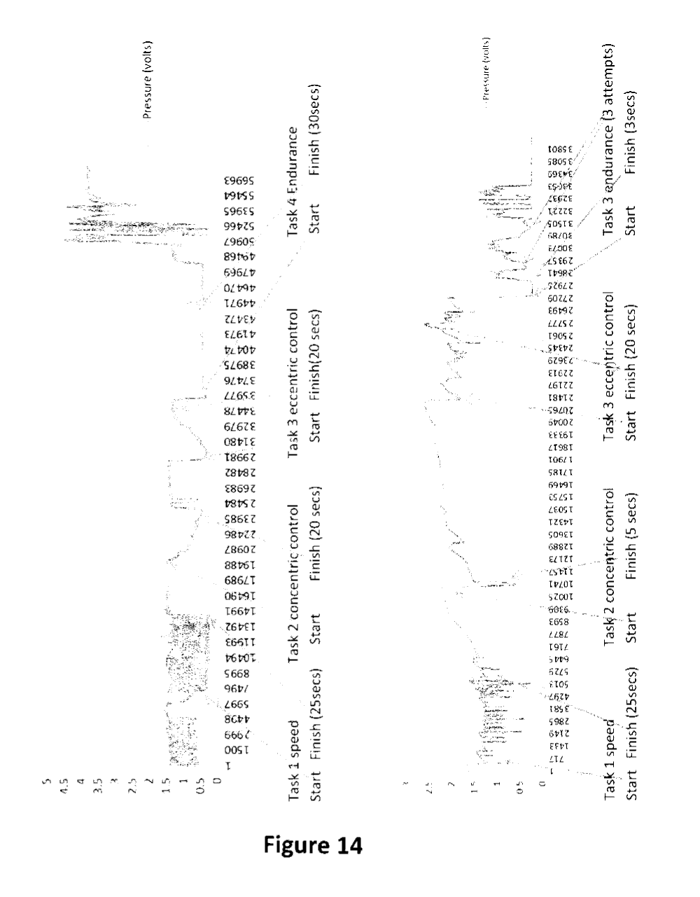

FIG. 14 schematically illustrates comparative screen displays for different participants where the user interface is in the form of a mobile device such as a mobile or cell phone. The screen display in this embodiment provides a continuous plot of pressure versus time for various predetermined tasks given to a participants with the intention of mobilizing their foot arch. The participants in these comparative examples are seated. The displayed signal has been inverted so that it is generally indicative of the foot arch height. In this example the tasks in order and as marked from left to right in FIG. 14 are as follows:
1. Speed task where a rapid sequence of elevations and lowers in the foot arch are performed over a continuous period, for example 20 to 30 seconds;
2. Concentric motor control task where a slow and controlled elevation in the foot arch is followed by a relatively fast lowering in the foot arch over a continuous period, for example 20 to 30 seconds;
3. Eccentric motor control where a slow and controlled lowering in the foot arch is followed by a relatively fast elevation in the foot arch over a continuous period, for example 20 to 30 seconds;
4. Endurance task where the foot arch is elevated and held in that position for an extended period such as a minimum of 20 seconds or failure or up to a maximum of 3 minutes.

These are exemplary foundation exercises that can be performed in different stances such as seated, double leg standing, and single leg standing. The double and single leg standing exercises increase the load down through the foot and make the exercises more challenging and increase resistance thereby improving foot muscle activation and strength. There are further and supplementary exercises which may be performed using the device such as individual great toe, lesser toe or heel lifts for improved foot motor control.

It will be understood that the device 1 in strengthening and extending the range of movement in the foot arch can function effectively as a therapeutic device. The participant may be graded for each task depending on the quality of the task completed as measured or derived from the output device of the biofeedback unit or device. The device 1 may also provide a diagnostic purpose in identifying flaws or weaknesses in motor control and other muscle functions associated with the foot. For example, in comparing the continuous plots of FIG. 14 it can be seen that:
1. the first participant in the upper graph has excellent control in their foot arch whereas the second participant in the lower graph has very poor control illustrated best by their failure to complete the tasks;
2. the first participant has high-functioning feet with no deformities, problems or foot pain and the results show good flexibility and intrinsic muscle strength and control;
3. the second participant has hyper-mobile (floppy) pronate feet and hallux valgus (bunion) on their great toe with the results indicating very poor intrinsic foot muscle tone and low control.

It will be appreciated that by observing this feedback substantially in real• time, the user will rapidly gain an awareness of the effect their intrinsic muscle activity has on their foot arch configuration. Over time, the user thereby gains a substantial degree of conscious control over their foot arch configuration, even if much of the sensation and muscle control in the foot has previously been lost. This awareness and control will of course be developed and strengthened through repetitive exercise of the foot muscles in combination with observation of the real-time feedback signals.

In one alternative embodiment, a change in the positional relationship between the heel and toes can be detected by the position sensors, and used as an indication of the change in configuration of the foot arch. In such embodiments, the contact sensors are used essentially to perform the function of the sensing means (i.e. the biofeedback sensor). In this case, the position sensors are configured and arranged not only to verify whether the foot is positioned correctly, but also to provide a qualitative or quantitative indication of any change in the positional relationship between the heel and toes, in response to foot muscle activity. Thus, in this embodiment, it will be appreciated that changes in the relationship between the heel and toes are used as a proxy for corresponding changes in the configuration of the foot arch.

The bladder 16 may be divided so that it includes multiple and separate pockets each designed to detect changes in pressure by contact with different areas of the foot arch. Alternatively the biofeedback sensor may take the form of separate bladders dispersed across and thus dedicated to different areas of the foot arch. The biofeedback sensor need not be limited to an inflatable bladder and may include other devices such as pressure pads which provide an electronic signal proportional to contact pressure with the foot arch.

In yet other embodiments, a combination of biofeedback and position sensors can be arranged to provide feedback to the user indicative of changes in configuration of the foot arch, in conjunction with changes in the positional relationship between the heel and toes, in response to foot muscle activity.

It has been found particularly that the provision of a direct feedback mechanism which ensures the foot is retained in the desired position, allows the user more effectively and consistently to isolate and control their intrinsic foot muscles in order to change the configuration of their foot arch. Otherwise, it can be difficult to distinguish between the influence of intrinsic foot muscle and extrinsic leg muscle activity, and therefore difficult to isolate the intrinsic foot muscles effectively. The degree of control a user has over their intrinsic foot muscles will develop over time through repetitive exercise of these muscles. The combination of repetitive exercise and real-time feedback of the effect that intrinsic muscle activity has on foot arch lift and drop will further enhance awareness of foot arch configuration. This increased awareness of, control over and exercise of the intrinsic foot muscles can lead to substantial improvements in localised blood circulation, as well as overall posture, gait and mobility, particularly in those suffering from various systemic diseases and related foot problems, and other posture related conditions arising from conditions such as obesity.

Accordingly, the present invention, at least in its preferred embodiments, provides a safe, robust, effective easy to use and relatively inexpensive device for measuring muscle activation and mid-arch movement in a human foot. The device advantageously provides the user with substantially real-time biofeedback of their foot muscle movement and the effect these muscles have on their foot arch configuration. In particular, preferred forms of the invention enable arch spring or lift to be measured and evaluated. Such evaluation could be incorporated into rehabilitation programs and could advantageously be used in combination with a specific set of Pilates or similar exercises for the lower limbs and feet, with a view to improving dynamic upright posture, particularly for elderly adults or those suffering from long-term postural problems. In particular, the use of preferred embodiments of the present device in such an exercise program can advantageously lead to significant improvements in foot functional performance and lumbo-pelvic stability. The device is understood to improve fine muscle control in the correct recruitment of intrinsic and extrinsic foot muscles. The method and apparatus of the invention could also be used by elite athletes, for example, to develop specific foot muscle control and strength, adapted to optimise their performance in specific sports such as gymnastics, running or cycling. In these and other respects, the invention in its preferred embodiments represents a practical and commercially significant improvement over the prior art.

Although the invention has been described with reference to specific examples, it will be appreciated by those skilled in the art that the invention may be embodied in many other forms. The device in its mobile form may be permanently or temporarily fitted to a chair or wheelchair and this conveniently makes the device readily accessible for the aged and less mobile users. The device can be adapted for use with the hand, neck or pelvic floor and in these alternative implementations is of similar construction to the foot arch device including sensing means for detecting changes in the configuration of the hand, the neck, or the pelvic floor in response to associated muscle activity. All such variations and modifications are to be considered within the scope of the present invention.

1. A device for evaluating the mobility of a foot arch of a person, the device including:
a base for supporting a foot;
sensing means associated with the base and arranged to detect a change in configuration of the foot arch predominantly occurring in response to intrinsic foot muscle activity within the supported foot, the sensing means being in contact with the foot arch;
position sensing means associated with the base and independent of the sensing means, said position sensing means configured to detect whether the supported foot is in a desired functionally effective position relative to the base;
a feedback module operatively associated with the sensing means for providing feedback to the person indicative of the change in the configuration of the foot arch predominantly occurring in response to the intrinsic foot muscle activity within the foot.

2. A device as claimed in claim 1 wherein the sensing means is mounted on the base such that the sensing means is contacted by at least a portion of the foot arch.

3. A device as claimed in claim 2 wherein the sensing means is a biofeedback sensor.

4. A device as claimed in claim 3 wherein the biofeedback sensor includes an elastically deformable body disposed such that changes to the configuration of the foot arch cause a corresponding change to the configuration of the elastically deformable body.

5. A device as claimed in claim 4 wherein the elastically deformable body is an inflatable bladder.

6. A device as claimed in claim 5 wherein the bladder is arranged on the base such that at least a portion of the sole of the supported foot is in direct face-to-face abutment with an operatively upper surface of the bladder.

7. A device as claimed in claim 6 wherein the biofeedback sensor includes a pressure sensor for detecting changes in pressure within the bladder corresponding to changes in the configuration of the foot arch.

8. A device as claimed in claim 7 wherein the biofeedback sensor includes a pump for inflating the bladder to a predetermined pressure.

9. A device as claimed in claim 8 wherein the feedback module includes an output device for providing the feedback to the user.

10. A device as claimed in claim 9 wherein the output device provides the feedback as one or more output signals including one or more of audio, visual and/or tactile signals.

11. A device as claimed in claim 10 wherein the feedback module is in communication with the pressure sensor whereby the pressure detected by the pressure sensor is converted to the output signal.

12. A device as claimed in claim 9 wherein the output device has a display for displaying the feedback to the user as a visual signal.

13. A device as claimed in claim 12 wherein the visual signal is a variable signal which changes in shape and/or size in response to changes in the configuration of the foot arch.

14. A device as claimed in claim 1 wherein the position sensing means includes one or more pressure or contact sensors.

15. A device as claimed in claim 14 wherein the one or more sensors include a heel sensor and at least one toe sensor.

16. A device as claimed in claim 15 wherein the heel sensor and/or the at least one toe sensor are selectively moveable relative to one another to accommodate various sizes of feet, as well as both left and right feet.

17. A device as claimed in claim 1 also including a user interface comprising the feedback module and an input module.

18. A device as claimed in claim 17 wherein the user interface is connected to the sensing means via a control line for allowing communication between the sensing means, the feedback module and the input module.

19. A device as claimed in claim 17 wherein the user interface is in wireless communication with the sensing means.

20. A method of evaluating the mobility of a foot arch of a person, said method including the steps of:
supporting a foot of the person on a base;
providing sensing means associated with the base and arranged to detect a change in configuration of the foot arch predominantly occurring in response to intrinsic foot muscle activity within the supported foot, the sensing means being in contact with the foot arch;
providing position sensing means associated with the base and independent of the sensing means, said position sensing means configured to detect whether the supported foot is in a desired functionally effective position relative to the base; and
using a feedback module operatively associated with the sensing means to provide feedback to the person indicative of the change in the configuration of the foot arch predominantly occurring in response to the intrinsic foot muscle activity within the foot.
